(19) United States
(12) Patent Application Publication
(10) Pub. No.: US 2022/0280349 A1
(43) Pub. Date: Sep. 8, 2022

(54) SMART DIAPER SYSTEM AND METHODS FOR MAKING THE SAME

(71) Applicant: Qatar Foundation for Education, Science and community Development, Doha (QA)

(72) Inventors: Saleem Ali, Doha (QA); Amine Bermak, Doha (QA); Shawkat Khan, Doha (QA)

(21) Appl. No.: 17/687,048

(22) Filed: Mar. 4, 2022

Related U.S. Application Data
(60) Provisional application No. 63/157,034, filed on Mar. 5, 2021.

(51) Int. Cl. A61F 13/42; G16H 40/67; G16H 40/63; A61F 13/15

(52) U.S. Cl. CPC A61F 13/42; G16H 40/67; G16H 40/63; A61F 13/15577; A61F 2013/424; A61F 2013/421

(57) ABSTRACT

A smart diaper system and methods for making the same. The system includes a diaper, an embedded sensor array, fixed to the diaper, including at least one sensor configured to detect a stimuli and a wireless transmission module operably connected to the embedded sensor array, configured to transmit a signal to a peripheral device. Additionally, in response to detecting a stimuli, the embedded sensor array communicates with the wireless transmission module to transmit a signal to the peripheral device corresponding to the detected stimuli. The method for manufacturing a smart diaper system includes providing a diaper, providing an embedded sensor array, including at least one sensor configured to detect a stimuli. Additionally, the embedded sensor array consists of screen printed sensors printed directly on a thermal transfer substrate and the thermal transfer substrate featuring the embedded sensor array becomes a fixed integral part of the diaper.

### PRIORITY CLAIM

The present application claims priority to and the benefit of U.S. Provisional Patent Applications No. 63/157,034, filed on Mar. 5, 2021, the entirety of which is incorporated herein by reference.

### BACKGROUND

Diapers are essential and very important for infants and critical patients with urinary traction problems. However, continuous and longtime usage of diaper can cause health issues such as rashes and bacterial infections. Such problems can be avoided by using a smart diaper to monitor the real-time health conditions of the diaper wearer to provide comfort, and reduce embarrassment. A smart diaper is used to monitor real-time conditions of the wearer (infant or elderly) such as wetting level, body temperature, and respiration rate on a smartphone through Bluetooth technology.

The body temperature data is very important for patients dealing with various chronic diseases and continuous monitoring of the health condition gives a deeper insight into the health condition leading the medical experts for a more valuable analysis and conclusions. Wetting sensor provides freedom to the caregiver and reduces time consumption by checking the diaper visually for human waste. Wetting sensors reduce health related issues due to human waste such as rashes and bacterial infections. Real-time monitoring of respiration rate for infants and critical patients or elderly is very important to control sudden death. Respiration rate sensor provides details of the breathing pattern whether the person under observation is breathing too fast or too slow to get attentions of the care taker immediately.

Accordingly, a system for monitoring health metrics of a diaper wearer in real-time without user and/or caregiver intervention is desired.

### SUMMARY

The present disclosure provides a new and innovative system for monitoring health conditions of a diaper wearer in real-time without the need for caregiver intervention.

The provided system includes an absorbent diaper. The absorbent diaper is configured to include low-cost screen printed sensors arranged into a sensor array. The sensor array includes multiple sensors, each capable of detecting different health conditions and/or stimuli. The sensor array can either be embedded in the substrate of the diaper or selectively detectable from the diaper to allow for reuse of the sensor array. For example, the sensor array can be configured to detect the body temperature of the wearer, respiration rate of the wearer, presence of wetness, and/or presence of volatile organic compounds (VOCs) indicative of human waste. The provided system also includes a wireless transmission module configured to transmit a signal to a peripheral device such as a tablet, smartphone and/or laptop in response to a signal from the sensor array. Various wireless communication methods, such as Bluetooth protocols, can be utilized. This allows for a non-invasive and continuous wireless monitoring. The sensor array's location on the diaper is experimentally optimized to get the vital information correctly.

It has been shown that monitoring health metrics of a diaper wearer in real-time provides comfort, and reduce embarrassment to the diaper wearer. Also, monitoring critical health indicators, such as respiration rate, in real-time allows clinicians to be better informed and able to provide superior care to patients.

An additional benefit of the provided system includes reducing the demand on clinicians and caregivers. The present disclosure allows for real-time monitoring of a patient's health metrics without the need for continuous observation by a clinician and/or caregiver.

In light of the disclosure, and without limiting the scope of the invention in any way, in a first aspect of the present disclosure, which may be combined with any other aspect listed herein unless specified otherwise, a smart diaper system includes a diaper, an embedded sensor array, fixed to the diaper, including at least one sensor configured to detect a stimuli and a wireless transmission module operably connected to the embedded sensor array, configured to transmit a signal to a peripheral device. Additionally, in response to detecting a stimuli, the embedded sensor array communicates with the wireless transmission module to transmit a signal to the peripheral device corresponding to the detected stimuli.

In another aspect of the present disclosure, which may be used in combination with any other aspect or combination of aspects listed herein, the system includes the embedded sensor array that consists of screen printed sensors printed directly on a thermal transfer substrate, wherein the thermal transfer substrate featuring the embedded sensor array becomes integral part of the diaper.

In another aspect of the present disclosure, which may be used in combination with any other aspect or combination of aspects listed herein, the system includes the embedded sensor array that is bendable, foldable, and stretchable with conformal integration capability onto nonplanar surface.

In another aspect of the present disclosure, which may be used in combination with any other aspect or combination of aspects listed herein, the system includes the embedded sensor array that detects human body temperature.

In another aspect of the present disclosure, which may be used in combination with any other aspect or combination of aspects listed herein, the system includes the embedded sensor array that detects the presence and/or absence of volatile organic compounds present in the human waste.

In another aspect of the present disclosure, which may be used in combination with any other aspect or combination of aspects listed herein, the system includes the embedded sensor array that detects wetness of the diaper as an indication about amount of liquid inside the diaper.

In another aspect of the present disclosure, which may be used in combination with any other aspect or combination of aspects listed herein, the system includes the embedded sensor array that detects real-time respiration rate of the smart diaper wearer.

In another aspect of the present disclosure, which may be used in combination with any other aspect or combination of aspects listed herein, the system includes the wireless transmission module that is configured to be selectively detachable from the diaper.

In another aspect of the present disclosure, which may be used in combination with any other aspect or combination of aspects listed herein, the system includes the signal transmitted by the wireless transmission module to the peripheral device corresponding is a Bluetooth signal.

In another aspect of the present disclosure, which may be used in combination with any other aspect or combination of aspects listed herein, the system includes that the configuration of the embedded sensor array is customizable.

In light of the disclosure, and without limiting the scope of the invention in any way, in a first aspect of the present disclosure, which may be combined with any other aspect listed herein unless specified otherwise, a method for manufacturing a smart diaper, includes providing a diaper, providing an embedded sensor array, including at least one sensor configured to detect a stimuli, wherein the embedded sensor array consists of screen printed sensors printed directly on a thermal transfer substrate, wherein the thermal transfer substrate featuring the embedded sensor array becomes a fixed integral part of the diaper. The presently disclosed method also includes providing a wireless transmission module operably connected to the embedded sensor array, configured to transmit a signal to a peripheral device and operably connecting the embedded sensor array with the wireless transmission module to transmit a signal to the peripheral device corresponding to the detected stimuli.

In another aspect of the present disclosure, which may be used in combination with any other aspect or combination of aspects listed herein, the method includes the thermal transfer substrate featuring the embedded sensor array becomes integral part of the diaper.

In another aspect of the present disclosure, which may be used in combination with any other aspect or combination of aspects listed herein, the method includes the embedded sensor array is bendable, foldable, and stretchable with conformal integration capability onto nonplanar surfaces.

In another aspect of the present disclosure, which may be used in combination with any other aspect or combination of aspects listed herein, the method includes the embedded sensor array detects human body temperature.

In another aspect of the present disclosure, which may be used in combination with any other aspect or combination of aspects listed herein, the method includes the embedded sensor array detects the presence and/or absence of volatile organic compounds present in the human waste.

In another aspect of the present disclosure, which may be used in combination with any other aspect or combination of aspects listed herein, the method includes the embedded sensor array detects wetness of the diaper as an indication about amount of liquid inside the diaper.

In another aspect of the present disclosure, which may be used in combination with any other aspect or combination of aspects listed herein, the method includes the embedded sensor array detects real-time respiration rate of the smart diaper wearer.

In another aspect of the present disclosure, which may be used in combination with any other aspect or combination of aspects listed herein, the method includes the wireless transmission module is configured to be selectively detachable from the diaper.

In another aspect of the present disclosure, which may be used in combination with any other aspect or combination of aspects listed herein, the method includes the signal transmitted by the wireless transmission module to the peripheral device corresponding is a Bluetooth signal.

In another aspect of the present disclosure, which may be used in combination with any other aspect or combination of aspects listed herein, the method includes the configuration of the embedded sensor array is customizable.

### BRIEF DESCRIPTION OF THE FIGURES

The present disclosure will become more fully understood from the detailed description given herein below for illustration only and thus does not limit the present disclosure, wherein:

FIG. 1 illustrates a schematic diagram of a smart diaper worn by a person of the presently disclosed system, according to an embodiment of the present disclosure.

FIG. 2 illustrates a schematic diagram of the smart diaper of the presently disclosed system, according to an embodiment of the present disclosure.

FIG. 3A illustrates a block diagram of an electronic wireless communication module of the presently disclosed system, according to an embodiment of the present disclosure.

FIG. 3B illustrates a block diagram of the sensor array of the presently disclosed system, according to an embodiment of the present disclosure.

FIG. 4A illustrates a schematic diagram a temperature sensor of the presently disclosed system, according to an embodiment of the present disclosure.

FIG. 4B illustrates a cross-sectional view of the temperature sensor of the presently disclosed system, according to an embodiment of the present disclosure.

FIG. 5A illustrates a schematic diagram of the wetting sensor of the presently disclosed system, according to an embodiment of the present disclosure.

FIG. 5B illustrates a cross-sectional view of the wetting sensor of the presently disclosed system, according to an embodiment of the present disclosure.

FIG. 6A illustrates a schematic diagram of the respiration rate sensor of the presently disclosed system, according to an embodiment of the present disclosure.

FIG. 6B illustrates a cross-sectional view of the respiration sensor of the presently disclosed system, according to an embodiment of the present disclosure.

FIG. 7 illustrates a schematic diagram of the multi-sensor patch, connected to detachable module, of the presently disclosed system, according to an embodiment of the present disclosure.

FIG. 8 illustrates experimental result of respiration rate sensor in an embodiment.

### DETAILED DESCRIPTION

The present disclosure provides a new and innovative system for monitoring health conditions of a diaper wearer in real-time and methods of making the same.

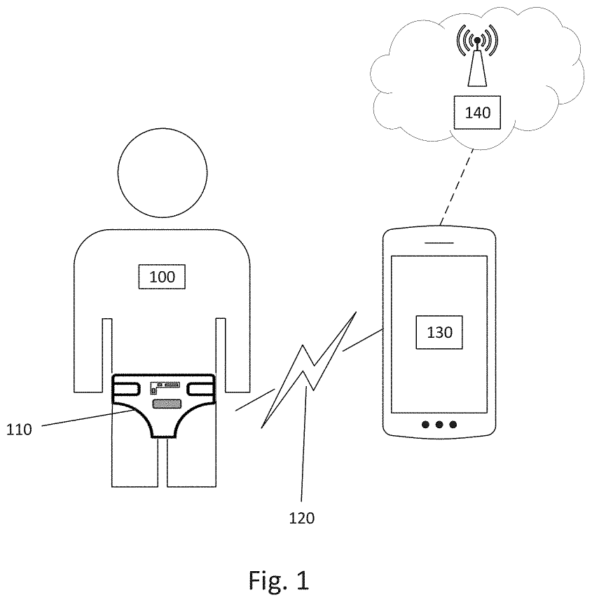

FIG. 1 illustrates a schematic diagram of a smart diaper worn by a person of the presently disclosed system, according to an embodiment of the present disclosure. Referring to FIG. 1, in an embodiment, the smart diaper system includes a smart diaper 110 worn by a person 100. The person 100 may be either an infant or an adult. In an embodiment, the smart diaper 110 includes a lower power wireless transmission module configured to transmit a signal 120 from the smart diaper 110 sensors to a peripheral device 130. The signal 120 may be any short-range wireless transmission protocol that utilizes radio signals to create a personal area network. For example, the signal 120 may be a Bluetooth® (BT) signal. In an additional embodiment, the signal 120 may be a ZigBee signal. In an embodiment, the peripheral device 130 may be a smartphone or any device capable of receiving a radio signal such as a tablet and/or laptop.

In an embodiment, the heath data of the smart diaper wearer 100 is transmitted to the peripheral device 130, which is utilized by a caregiver and displayed in real-time to ensure timely attention, and service can be given to the smart diaper wearer 100. Additionally, the techniques disclosed herein provide freedom of movement to the caregiver for moving around within the premises while monitoring the diaper wearer 100 in real-time given the range of signal 120. In an additional embodiment, the peripheral device 130 may be capable of connecting to a network 140, such as the internet.

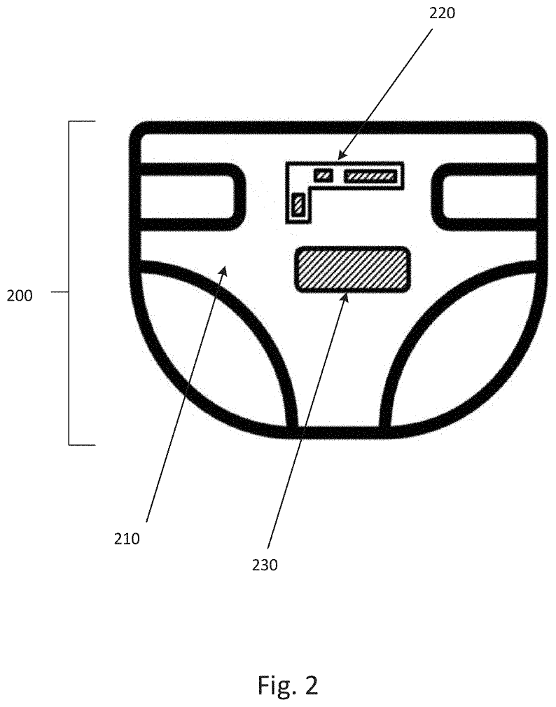

FIG. 2 illustrates a schematic diagram of the smart diaper of the presently disclosed system, according to an embodiment of the present disclosure. Referring to FIG. 2, the smart diaper 200 includes a diaper 210. For example, the diaper 210 may be any commercially available diaper such as a cloth diaper and/or synthetic disposable diaper. Additionally, the diaper 210 may be either an infant diaper, toddler diaper and/or adult diaper. In an embodiment, the smart diaper 200 includes a sensor array patch 220 and a wireless communication system 230. In an embodiment, individual sensors are screen printed directly on a thermal transfer substrate and subsequently transferred to the sensor array patch 220 through thermal lamination process. Additionally, the printed sensors become integral part of the diaper 210, which is bendable, foldable and stretchable with conformal integration capability onto nonplanar surfaces of the diaper 210. The sensor array patch 220 includes at least one sensor to detect a stimuli. In an embodiment, the sensor array patch 220 includes three different sensors targeted for monitoring multiple stimuli, such as, the temperature, respiration rate, and wetness of the diaper 210. The sensors are interconnected to the signal conditioning circuits to eliminate the redundant data generated through exposure of the sensors to the surrounding environment. Additionally, in an embodiment, the sensor array patch 220 is operably connected to the wireless communication system 230 to allow the stimuli detected by the sensor array patch 220 to be transmitted to the caregiver of the smart diaper 200 wearer.

In an embodiment, the smart diaper 200 has the characteristics to be worn easily given the printed materials are conformable to uneven surfaces. For example, the foldability and/or stretchability of the diaper 210 does not impact the overall performance of the sensor array patch 220. In an example, the sensor array patch 220 can be developed on a wide variety of substrates to provide additional surface area for the inclusion of additional sensors. For example, in an embodiment, sensor array patch 220 may include sensor for monitoring additional biomarkers such as VOCs (volatile organic compounds) present in the waste produced by the wearer of diaper 200 and collected in diaper 210.

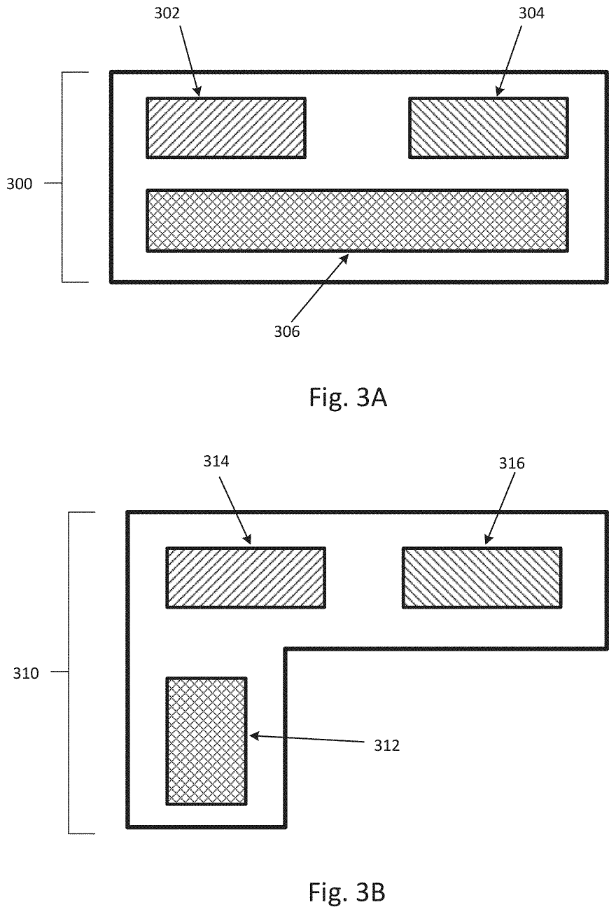

FIG. 3A illustrates a block diagram of an electronic wireless communication module of the presently disclosed system, according to an embodiment of the present disclosure. Referring to FIG. 3A, the wireless communication system 300 includes readout circuit 302, processing unit 304 and wireless transmission unit 306. In an embodiment, utilizing the wireless communication system 300, the smart diaper wearer's heath data is transmitted to the peripheral device of the caregiver and displayed in real-time to ensure timely attention to the specific needs of the wearer of the smart diaper. Additionally, the proposed technique utilizing the wireless communication system 300, provides freedom to the caregiver to allow the caregiver to move around while monitoring the diaper wearer in real-time. In an embodiment, the wireless communication system 300 of the diaper includes a local alarm to alert the caregiver via the peripheral device that the peripheral device, such as a smartphone, is silent or away from the caregiver. Additionally, the wireless communication system 300 includes a rechargeable battery to power the wireless communication system 300.

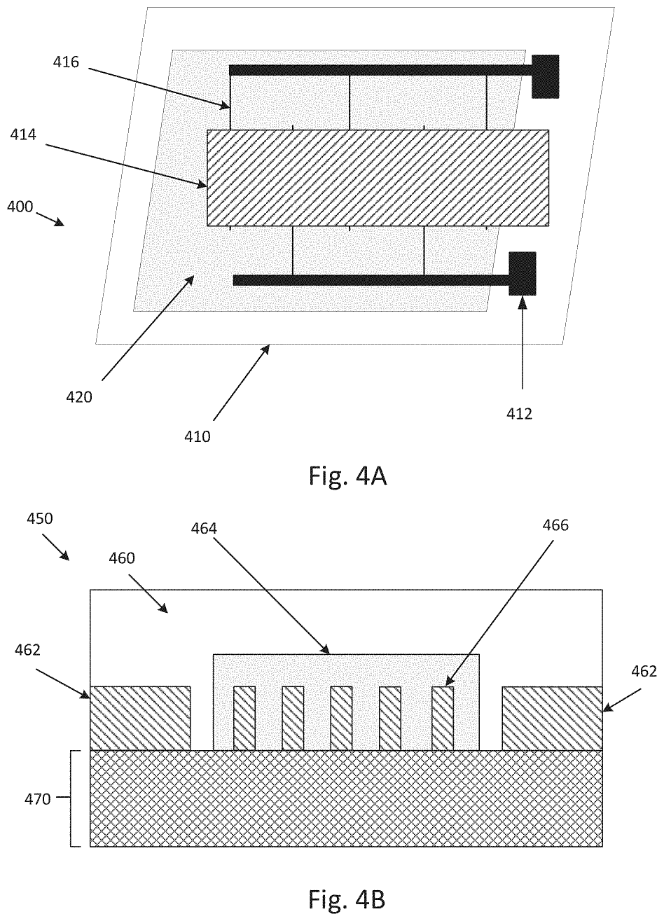

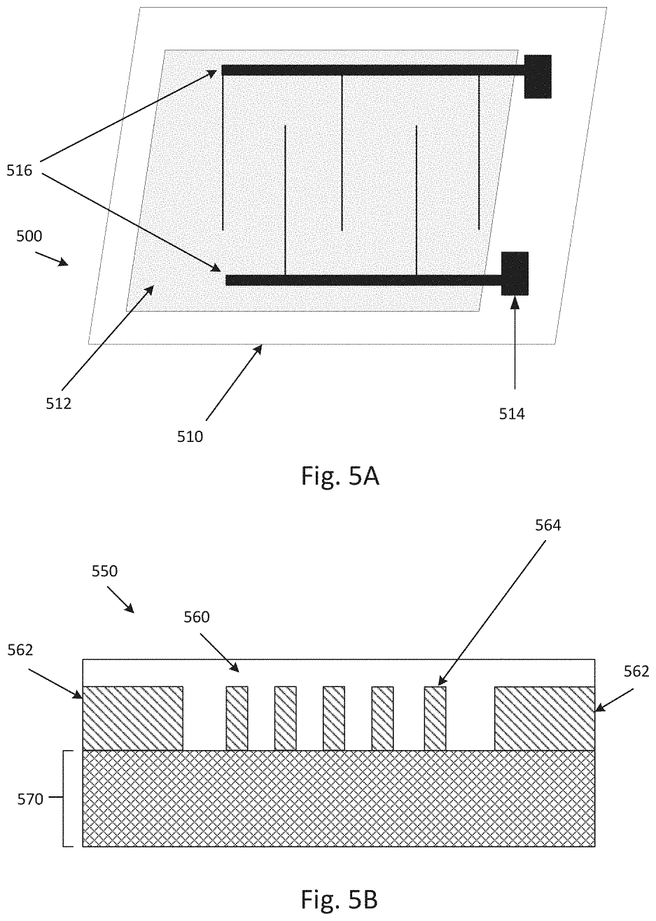

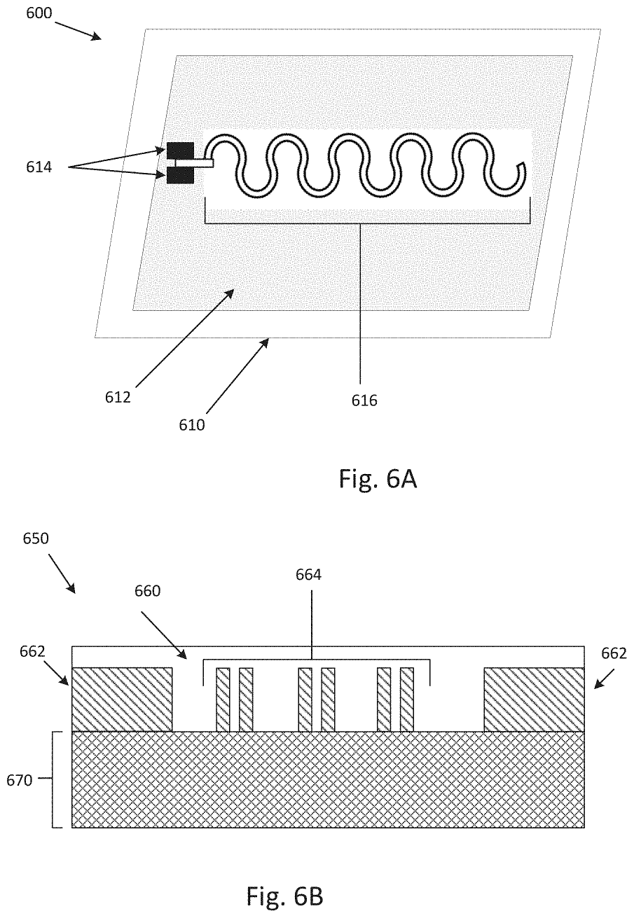

FIG. 3B illustrates a block diagram of the sensor array of the presently disclosed system, according to an embodiment of the present disclosure. Referring to FIG. 3B, in an embodiment, the sensor array 310 includes a temperature sensor 312 (FIG. 4A), wetting sensor 314 (FIG. 5A), and respiration rate sensor 316 (FIG. 6A). In an embodiment, the sensor array 310 may include various other sensors capable of detecting a stimuli. Additionally, the sensor array 310 is configured to operably communicate with the wireless communication system 300 of FIG. 3A.

FIG. 4A illustrates a schematic diagram a temperature sensor of the presently disclosed system, according to an embodiment of the present disclosure. Referring to FIG. 4A, in an embodiment, the temperature sensor 400 consists of substrate 410, conductive interdigital electrodes 416, sensing film 414, connecting pads 412 and encapsulation layer 420. The sensor 400 is developed by printing conducting interdigital electrodes (IDEs) 416 and filled with the temperature sensing layer 414. An equal spacing between the electrodes 416 is maintained to ensure containment of the sensing layer 414 and exposure to the detection event without being interrupted by the surrounding environment. The interconnections are also printed by using the same ink for the readout port. A thin encapsulant layer 420 is applied on the entire device including interconnect and sensing layer 414. The encapsulant layer 420 plays a role in the device performance, stability, endurance, and wearer comfort as printed devices are processed at low temperature and fragile when left uncovered.

In an embodiment, body temperature data is important for patients dealing with various chronic diseases and continuous monitoring of the health conditions. Additionally, body temperature data provides a deep insight into the health condition providing the medical experts with more valuable analysis and conclusions.

FIG. 4B illustrates a cross-sectional view of the temperature sensor of the presently disclosed system, according to an embodiment of the present disclosure. Referring to FIG. 4B, the sensor 450 includes a substrate 470, connecting pads 462, interdigital electrodes 466, sensing film 464 and encapsulation layer 460.

FIG. 5A illustrates a schematic diagram of the wetting sensor. Referring to FIG. 5A, in an embodiment, the wetting sensor 500 includes a substrate 510, interdigital electrodes 516, connecting pads 514, encapsulating film 512. In an embodiment, the sensor 500 structure is composed of interdigital electrodes 516 without any sensing layer. Additionally, conductive ink is patterned using screen printing techniques. In an embodiment, the spacing between the electrodes 516 is optimized to get the maximum sensitivity to variations in the wetting level of the diaper utilizing sensor 500. In presence of liquid in the diaper, interdigital electrode 516 experience conductivity changes and produce a change in the electrical resistance, which is correlated with quantity of liquid. The change in electrical properties of sensor 500, indicating wetness of the smart diaper, is communicated via the wireless communication system previously disclosed above.

In an embodiment, the wetting sensor 500 provides freedom of movement to the caregiver and reduces time needed to check the diaper visually for wetness which is indicative of human waste. Additionally, wetting sensors reduce health related issues due to human waste such as rashes and bacterial infections.

FIG. 5B illustrates a cross-sectional view of the wetting sensor of the presently disclosed system, according to an embodiment of the present disclosure. Referring to FIG. 5B, the wetting sensor 550 includes as substrate 570, connecting pads 562, and interdigital electrodes 564 and encapsulating film 560.

FIG. 6A illustrates a schematic diagram of the respiration rate sensor of the presently disclosed system, according to an embodiment of the present disclosure. Referring to FIG. 6A, the respiration rate sensor 600 includes a substrate 610, a horseshoe sensing pattern 616, connecting pads 614 and encapsulating layer 612. Additionally, the respiration sensor 600 is patterned in a horse-shoe shape 616 using a stretchable substrate 610 that allows impregnation with the conductive ink. In an embodiment, the sensor 600 works similar to a strain sensor, where the change in electrical resistance is exploited as a detection of force application. The slight variation due to the instantaneous pressure applied as a result of breathing, is used to detect the respiration rate. The resistance modulation phenomenon is exploited for the breath detection and respiration rate sensing applications. The change in electrical properties of sensor 600, indicating respiration rate of the smart diaper wearer, is communicated via the wireless communication system previously disclosed above.

In an example, the respiration sensor 600 provides breath data and the respiration rate of a person under observation. For example, real-time monitoring of respiration rate for infants and critical patients or elderly is very important to control sudden death. Respiration rate sensor 600 provides details of the breathing pattern of the person under observation and can determine if the breathing is too fast or too slow and, if either apply, to get attentions of the care taker.

FIG. 6B illustrates a cross-sectional view of the respiration sensor of the presently disclosed system, according to an embodiment of the present disclosure. Referring to FIG. 6B, in an embodiment, the respiration rate sensor 650 includes the substrate 670, connecting pads 662, sensing pattern 664 and encapsulating layer 660.

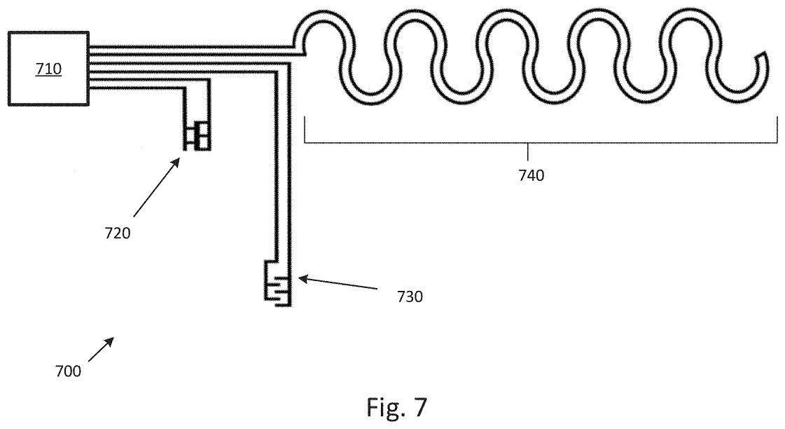

FIG. 7 illustrates a schematic diagram of the multi-sensor patch, connected to detachable module, of the presently disclosed system, according to an embodiment of the present disclosure. Referring to FIG. 7, in an embodiment, the multi-sensor patch 700 includes a temperature sensor 720, a wetting level sensor 730, and respiration rate sensor 740. In an embodiment, sensors 720, 730 and 740 are integrated onto the diaper, whereas the detachable module 710 is not integrated onto the surface of the diaper. Additionally, the detachable module 710 is connected to the connection pads of the connecting port of the sensors patches 720, 730 and 740 through flexible zero insertion force (ZIF) connectors. The detachable module 710 can be reversibly attached on the front side of the smart diaper to minimize discomfort of the wearer in case of posture changing.

In an embodiment, the detachable module 710 may be the wireless communication system 300 in FIG. 3A. Additionally, the detachable module 710 includes the readout, signal processing and wireless transmission circuits. The detachable module 710 includes a readout and a filtration circuit, power management and charging components, a microcontroller for signal conditioning and processing, and a low power Bluetooth unit. All of the sensors featured on the sensor array are operably connected to the microcontroller and data is and processed in real time.

In an example, the detachable module 710 allows the diaper to be replaced while not having to replace the detachable communication module 710. Additionally, the detachable communication module 710 is able to be reconnected to the sensor array patch after charging the power battery. In an embodiment, the portable nature of the detachable module 710 and fast data processing carried out by the system allows for minimal interruption of patient monitoring by the disclosed system. These features, among several other, presents a valuable contribution in real-time health monitoring systems.

In an embodiment, the materials used in the smart diaper system are biocompatible and do not pose any threats or harm to the wearer's health. Additionally, the sensor array patch and the interconnections between components are encapsulated with thin plastic thin film to protect the printed sensors and to protect the human wearer from the electricity within the electronic sensors.

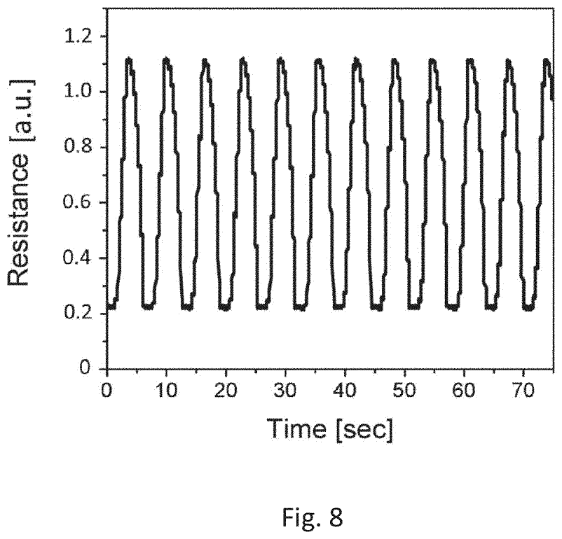

FIG. 8 illustrates experimental results of the respiration cycles recorded by connecting the sensor to a sourcemeter and recording real-time data. The strain sensor detects application of force, wherein change in force applied translates to electrical resistance [a.u.] charted over time in 10 second intervals.

The present disclosure provides a method for manufacturing a smart diaper. The provided method includes providing a diaper, providing an embedded sensor array, including at least one sensor configured to detect a stimuli. The embedded sensor array consists of screen printed sensors printed directly on a thermal transfer substrate and the thermal transfer substrate featuring the embedded sensor array becomes a fixed integral part of the diaper. Additionally, the method includes providing a wireless transmission module operably connected to the embedded sensor array, configured to transmit a signal to a peripheral device. The presently disclosed method also includes operably connecting the embedded sensor array with the wireless transmission module to transmit a signal to the peripheral device corresponding to the detected stimuli.

Certain non-limiting embodiments and figures of the present disclosure are further disclosed in the document entitled “Smart Diaper Information” and submitted herewith as Exhibit A.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

The invention is claimed as follows:

1. A smart diaper system comprising:
a diaper;
an embedded sensor array, fixed to the diaper, including at least one sensor configured to detect a stimuli; and
a wireless transmission module operably connected to the embedded sensor array, configured to transmit a signal to a peripheral device;
wherein, in response to detecting a stimuli, the embedded sensor array communicates with the wireless transmission module to transmit a signal to the peripheral device corresponding to the detected stimuli.

2. The smart diaper according to claim 1, wherein the embedded sensor array consists of screen printed sensors printed directly on a thermal transfer substrate, wherein the thermal transfer substrate featuring the embedded sensor array becomes integral part of the diaper.

3. The smart diaper according to claim 1, wherein the embedded sensor array is bendable, foldable, and stretchable with conformal integration capability onto nonplanar surface.

4. The smart diaper according to claim 1, wherein the embedded sensor array detects human body temperature.

5. The smart diaper according to claim 1, wherein the embedded sensor array detects the presence and/or absence of volatile organic compounds present in the human waste.

6. The smart diaper according to claim 1, wherein the embedded sensor array detects wetness of the diaper as an indication about amount of liquid inside the diaper.

7. The smart diaper according to claim 1, wherein the embedded sensor array detects real-time respiration rate of the smart diaper wearer.

8. The smart diaper according to claim 1, wherein the wireless transmission module is configured to be selectively detachable from the diaper.

9. The smart diaper according to claim 1, wherein the signal transmitted by the wireless transmission module to the peripheral device corresponding is a Bluetooth signal.

10. The smart diaper according to claim 1, wherein the configuration of the embedded sensor array is customizable.

11. A method for manufacturing a smart diaper, the method comprising:
providing a diaper;
providing an embedded sensor array, including at least one sensor configured to detect a stimuli,
wherein the embedded sensor array consists of screen printed sensors printed directly on a thermal transfer substrate,
wherein the thermal transfer substrate featuring the embedded sensor array becomes a fixed integral part of the diaper,
providing a wireless transmission module operably connected to the embedded sensor array, configured to transmit a signal to a peripheral device,
operably connecting the embedded sensor array with the wireless transmission module to transmit a signal to the peripheral device corresponding to the detected stimuli.

12. The method of claim 11, wherein the thermal transfer substrate featuring the embedded sensor array becomes integral part of the diaper.

13. The method of claim 11, wherein the embedded sensor array is bendable, foldable, and stretchable with conformal integration capability onto nonplanar surfaces.

14. The method of claim 11, wherein the embedded sensor array detects human body temperature.

15. The method of claim 11, wherein the embedded sensor array detects the presence and/or absence of volatile organic compounds present in the human waste.

16. The method of claim 11, wherein the embedded sensor array detects wetness of the diaper as an indication about amount of liquid inside the diaper.

17. The method of claim 11, wherein the embedded sensor array detects real-time respiration rate of the smart diaper wearer.

18. The method of claim 11, wherein the wireless transmission module is configured to be selectively detachable from the diaper.

19. The method of claim 11, wherein the signal transmitted by the wireless transmission module to the peripheral device corresponding is a Bluetooth signal.

20. The method of claim 11, wherein the configuration of the embedded sensor array is customizable.
